(19) United States
(12) Patent Application Publication
(10) Pub. No.: US 2003/0203881 A1
(43) Pub. Date: Oct. 30, 2003

(54) Method of treating neurologic disorders

(75) Inventor: Ian D. Duncan, Madison, WI (US)

Correspondence Address: MICHAEL BEST & FRIEDRICH, LLP, ONE SOUTH PINCKNEY STREET, P O BOX 1806, MADISON, WI, 53701

(73) Assignee: NeuroTherapeutics LLC, Madison, WI

(21) Appl. No.: 10/383,115

(22) Filed: Mar. 6, 2003

Related U.S. Application Data
(63) Continuation-in-part of application No. PCT/US01/27593, filed on Sep. 6, 2001.
(60) Provisional application No. 60/230,350, filed on Sep. 6, 2000.

(51) Int. Cl. A61K 31/65

(52) U.S. Cl. 514/152

(57) ABSTRACT

The invention provides a method of treating certain neurological diseases by administering to a patient in need thereof an effective amount of a tetracycline compound.

### CROSS-REFERENCE TO RELATED APPLICATIONS

This application claims the benefit of priority from International Application No. PCT/US01/27593, filed Sep. 6, 2001, which claims priority to U.S. Provisional Application 60/230,350, filed Sep. 6, 2000, which is incorporated herein by reference.

### STATEMENT REGARDING FEDERALLY SPONSORED RESEARCH OR DEVELOPMENT

Not Applicable

This invention relates to a method of treating neurologic disorders including Alzheimer's disease, Guillain Barré Syndrome, adreneoleukodystrophy, Parkinson's disease, and amyotrophic lateral sclerosis, by administering an effective amount of a tetracycline compound.

Alzheimer's disease is probably the best known central nervous system (CNS) disorder. It is the most prevalent human neurodegenerative disease and creates untold human suffering at a huge social and economic cost worldwide. The cause of the disease is unknown and there are no cures nor, indeed, treatments that in any way ameliorate the disease course. Affected patients develop classic brain changes with the formation of amyloid plaques, neurofibrillary tangles, inflammation and degeneration of neurons. As a result of these changes, patients develop progressive memory loss that leads to dementia and total reliance on others.

While there have been recent advances in experimental therapies for Alzheimer's disease, none is close to being used in human treatment. Thus, there is a critical need for the discovery of new drugs or new applications of existing drugs to treat devastating, chronic neurologic disorders such as Alzheimer's disease.

One proposed therapeutic approach is to minimize or prevent the inflammatory changes that occur in the brains of, e.g., Alzheimer patients (Selkoe 1999). It is thought that a key cell in the cellular cascade that results in neuronal death in Alzheimer's disease is the microglial cell. These cells are associated with amyloid plaques and produce a number of pro-inflammatory cytokines that can result in neuronal death. Thus, use of anti-inflammatory agents may be of value in the treatment of neurologic disorders.

Second generation tetracycline drugs, well-known for their antibiotic effects, also have known anti-inflammatory effects and could have therapeutic use in many serious central nervous system (CNS) disorders.

The present invention provides a method of treating neurologic disorders which includes administering an effective amount of a tetracycline compound of formula (I), described hereinafter. In another aspect, the invention provides a method of providing to a mammalian system an effective amount of a tetracylcine compound sufficient to down regulate microglia expression, activation and production, and hence, prevent, reduce or minimize inflammation.

A fuller appreciation of the specific attributes of this invention will be gained upon an examination of the detailed description of preferred embodiments and appended claims.

This invention relates to a method of treating neurologic disorders such as Alzheimer's disease, Guillain Barré Syndrome, adreneoleukodystrophy, Parkinson's disease, and amyotrophic lateral sclerosis, by administering an effective amount of a tetracycline compound. The present invention is directed to the prevention or treatment of a broad spectrum of diseases, which may be linked to microglial activation and inflammation. The method can be used to prevent, inhibit or alleviate any condition in which results from upregulation of expression or activity of microglia.

In one aspect, a tetracycline compound suitably in accordance with the present invention is set forth in formula (I):

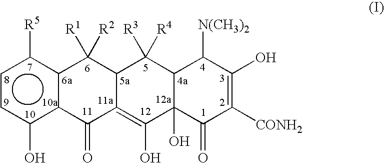

wherein R¹ is CH₃ or OH and R² is H or OH, or R¹ and R² taken together are ═CH₂, R³ and R⁴ are H or OH and R⁵ is Cl or N(CH₃)₂. Compounds of formula (I) of particular value in accordance with the present invention are minocycline and doxycycline, i.e., wherein R¹, R², R³ and R⁴ are H and R⁵ is N(CH₃)₂; and R¹ is CH₃, R² is H, R³ is OH, and R⁴ and R⁵ are H, respectively. Compounds of formula (I) generally have antibiotic activity.

However, non-antibiotic tetracyclines, i.e., those that have little or no antibiotic activity, are also contemplated within the scope of the method in accordance with the present invention. Mitscher (1978) has reviewed the modifications to the basic tetracycline structure and their effect on retention of antibiotic properties. According to Mitscher, modifications at positions 5-9 of the tetracycline ring system can be made without causing the complete loss of antibiotic properties. However, changes to the basic structure of the rings system, or replacement or deletion of substituents at position 1-4 or 1-12, generally lead to synthetic tetracyclines with substantially less, or essentially no, antibacterial, antimicrobial, i.e., non-antibiotic, activity. The compounds are often referred to as chemically modified tetracyclines or CMTs. Many CMTs have been synthesized. For example, 4-dedimethylaminotetracycline, i.e., deletion of the N(CH₃)₂ group at the C-4 position, is commonly considered to be a non-antibacterial, non-antimicrobial tetracycline.

Other examples of such tetracyclines include 6-demethyl-6-deoxy-4-dedimethylaminotetracycline, 4-dedimethyl-5-oxytetracycline, 4-hydroxy-4-dedimethylaminotetracycline, 5α,6-anhydro-4-hydroxy-4-dedimethylaminotetracycline, 6α-deoxy-5-hydroxy-4-dedimethylaminotetracycline, 4-dedimethylaminotetracycline, 4-dedimethylamino- 12α-deoxytetracycline, 6α-deoxy-5-hydroxy-4-dedimethylaminotetracycline, tetracyclinonitrile, 6α-benzylthiomethylenetetracycline, mono-N-alkylated amide of tetracycline, 6-fluoro-6-demethyltetracycline, and 11α-chlorotetracycline.

Thus, compounds of formula (I) as well as compounds in which the basic tetracycline structure has been altered to render a compound with little or no antibiotic activity are suitable in accordance with the present invention.

During recent years, it has been established that tetracyclines, which are rapidly absorbed and have a prolonged half-life, exert biological effects independent of their antimicrobial activity (Golub et al. 1991; Golub et al. 1992; Uitto et al. 1994). Such effects include inhibition of matrix metalloproteinases, including collagenase gelatinase and stromelysin which have been implicated in rheumatoid arthritis. These metalloproteinases are known to be upregulated in osteoarthritis-affected joints (Greenwald 1994; Mohtai et al. 1993). It has also been shown that prophylactic administration of doxycycline (a semisynthetic tetracycline) markedly reduced the severity of osteoarthritis in dog models (Yu et al (1992)), and that minocycline (a semisynthetic tetracycline) is safe and effective for patients with mild and moderate arthritis (Tilley et al. 1995). More, recent studies have also suggested that tetracyclines and inhibitors of metalloproteinases inhibit tumor progression (DeClerck et al. 1994), bone resorption (Rifkin et al 1994) and angiogenesis (Maragoudakis et al. 1994) and have anti-inflammatory properties (Ramamurthy et al. 1994).

It has been reported that minocycline, a tetracycline compound, reduced the microglial response in the area surrounding areas of brain ischemia in an experimental model of stroke, i.e., around stroke lesions, and that it protects neurons against death in tissue culture (Yijänheikki et al. 1999, 1998). It remains, however, unclear in other CNS disease states whether minocycline has any direct effect on microglial activation or may have a secondary effect to general inhibition of CNS inflammation. While CNS disease states appear to have some similarities, each has a very different mechanism or pathway to the clinical symptoms which manifest irreversible neuronal loss in specific regions of the brain. For example, in Parkinson's disease, loss of neurons from structures of the basal ganglia results in abnormalities in the control of movement. In Alzheimer's disease, loss of hippocampal and cortical neurons leads to impairment of memory and cognitive ability. In amyotrophic lateral sclerosis, muscle weakness results from the degeneration of spinal, bulbar and cortical motor neurons. Thus, a most striking characteristic of the CNS diseases is the exquisite specificity of the disease processes for particular types of neurons. The intrinsic physiological characteristics, among other influences, genetic and environmental, of the affected neurons are significant factors involved in disease onset and progression. There is regional variation, e.g., in the capacity for oxidative metabolism, i.e., the capacity to scavenge toxic free radicals in oxidative stress. Thus, because of selective neuronal vulnerability in the neurodegenerative disease, agents that may slow progression of or are neuroprotective in one disease does not necessarily transfer to another disease.

It has now been surprisingly and unexpectedly found that tetracyclines may be of value in treating certain neurologic disorders, such as Alzheimer's disease, Guillain-Barré Syndrome, Parkinson's disease, adrenoleukodystrophy and amyotrophic lateral sclerosis. The amount of the tetracycline compound used according to the present invention is an amount that is effectively inhibitory of microglial expression or activity. An amount of a tetracycline compound is effectively inhibitory of or downregulates microglia activity if it significantly reduces microglia expression or activity, or if it reduces microglial cell production. The amount is suitably an anti-inflammatory effective amount.

The tetracycline compounds useful in accordance with the present invention appear to exhibit their beneficial effect in a dose-dependent manner. Thus, within broad limits, administration of larger quantities of a tetracycline compound is expected to inhibit microglia activation to a greater degree than does administration of a smaller amount. Moreover, efficacy is also contemplated at dosages below the level at which toxicity is seen. Further, in practice, higher doses of the compounds of the present invention are generally used where the therapeutic treatment of a disease state is the desired end, while the lower doses are generally used for prophylactic purposes.

It will be appreciated that the specific dosage administered in any given case will be adjusted in accordance with the specific compounds being administered, the disease to be treated, the condition of the subject and other relevant medical factors that may modify the activity of the drug or the response of the subject, as is well known by those skilled in the art. For example, the specific dose for a particular patient depends on age, body weight, general state of health, on diet, on the timing and mode of administration, on the rate of excretion, and on medicaments used in combination and the severity of the particular disorder to which the therapy is applied. Dosages for a given patient can be determined using conventional considerations, e.g., by customary comparison of the differential activities of the subject compounds and of a known agent, such as by means of an appropriate conventional pharmacological protocol.

The maximal dosage for a subject is the highest dosage, which does not cause undesirable or intolerable side effects. For example, it is contemplated that doses from about 0.1 mg/kg/day to about 45 mg/kg/day, and suitably, from about 1 mg/kg/day to about 18 mg/kg/day are generally effective. Such dosages are generally considered below the antibiotic effective dose, typically about 45 mg/kg/day. Treatment would be given for about 2-3 weeks or until full recovery or until the patient becomes asymptomatic. It is anticipated that dosages of the tetracycline compound in accordance with the present invention will reduce symptoms at least 50% compared to pre-treatment symptoms.

Systemic administration of tetracycline compounds is contemplated in accordance with the present invention, especially those tetracycline compounds capable of substantial absorption and effective systemic distribution.

A particular pharmaceutical composition for use in the method in accordance with the present invention includes a combination of the tetracycline compound in a suitable pharmaceutical carrier (vehicle) or excipient as understood by practitioners in the art. Parenteral administration (e.g., intravenous injection) is a desirable route of delivery of the tetracycline. For parenteral application, particularly suitable are injectable, sterile solutions, oily or aqueous solution, as well as suspensions, emulsions, or implants, including suppositories. Ampoules are convenient unit dosage forms. The compositions for administration may include the tetracycline compound with appropriate diluents, carriers, and the like which are readily formulated.

Enteral administration (e.g., oral administration) is also desirable, and formulations such as tablets, liquids, drops, lozenges or capsules, can be employed to provide the compound. A syrup, elixir or the like can be used if a sweetened vehicle is desired.

Alternatively, delivery can by sustained, delayed release (i.e., controlled release) to provide a constant serum level of the tetracycline compound. Many controlled release systems for controlling the release of an active ingredient over the course of several hours are known, e.g., the wax matrix system, the coated granular, the “miniature osmotic pump” system and the Forest Synchron System of Forest Laboratories.

Also included within the scope of the present invention is the co-administration of the tetracycline compound in accordance with the present invention with one or more adjunct agent capable of inhibiting inflammation or relieving oxidative stress in tissue, e.g., steroidal and non-steroidal anti-inflammatory drugs or free radical scavengers. The term “co-administration” includes administration of two or more agents in a single unitary dosage form, administration of agents simultaneously (i.e., at the same time), and administration of agents sequentially. It is anticipated that a compound of formula (I) used in combination with various anti-inflammatory agents can give rise to a significantly enhanced anti-inflammatory effect, thus providing an increased therapeutic effect. Specifically, as a significantly increased anti-inflammatory effect is obtained with the above disclosed combinations utilizing lower concentrations of the anti-inflammatory drugs compared to treatment regimes in which the drugs are used alone, there is the potential to provide therapy wherein adverse side effects associated with anti-inflammatory agents are considerably reduced than normally observed with the anti-inflammatory agents used alone in larger doses.

It is also anticipated that an envisioned therapeutic effect can be obtained by co-administration of a compound of formula (I) and a free radical scavenger, e.g., tocopherol or a monoamine oxidase (MAO) inhibitor such as selegiline, wherein adverse side effects, including toxicity, are considerably reduced from those normally observed when these agents are used alone in high doses.

It is contemplated that the compounds of formula (I) and the foregoing agents may be packaged as a pharmaceutical package with a plurality of containers. At least one of the containers contains one or more of a compound of formula (I). Another container suitably contains an anti-inflammatory agent. Yet another container may contain a free radical scaventger. The packaging may contain one or both of the anti-inflammatory agent or free radical scavenger. Also included in the packaging are instructions for co-administration of all agents to a subject having Alzheimer's disease, Guillain Barré syndrome, Parkinson's disease, adrenoleukodystrophy or amyotrophic lateral sclerosis.

The following examples are provide to assist in a further understanding of the invention. The particular materials and conditions employed are intended to be further illustrative of the invention and are not limiting upon the reasonable scope thereof.

### EXAMPLE 1

### Alzheimer's Disease

A first set of experiments involves a model system which is a transgenic mouse that overexpresses (i.e., produces a gene product that exceeds levels of production in normal or non-transformed organisms) a mutant form of the human amyloid-β precursor protein. Two sets of experiments are performed.

In the first, the transgenic mice are treated with a tetracycline compound, e.g., minocycline (45 mg/kg/day) from the onset of pathological change for a period of 2-3 months and then the brains of the treated mice are studied morphologically. Differences evaluating inflammation, plaque formation and neuronal death are determined between this group and those receiving no treatment.

In a second set of experiments, a group of treated animals are studied for a shorter period of time as to the ability to resolve pathological changes after they have been present for some time.

Another set of experiments is performed utilizing an in vitro system. A protocol, established by Coombs et al (2000), is used in which microglia are added to neurons in culture and to which amyloid-62 peptides are added. Minocycline is then be added to these cultures and the expression of cytokines known to kill neurons is evaluated as is the survival of these cells in the same culture system. This experimental approach allows manipulation of variables in the system (i.e. dosage, other drugs) and the data generated is applicable to in vivo experimental models and, of course, therapeutic trials in humans.

### EXAMPLE 2

### Guillain Barré Syndrome (GBS)

Guillain Barré Syndrome is a neuropathy that shares many pathologic hallmarks with experimental allergic encephalomyelitis (EAE) except that inflammation, demyelination and degeneration are limited to the peripheral nervous system (PNS). Like EAE , an animal model exists which is called experimental allergic neuritis (EAN). It has been reported that, in EAE, minocycline prevents the invasion of inflammatory cells into the CNS. The goal in EAN will be to stop macrophages from infiltrating into the PNS. Such an approach is contemplated to have important therapeutic applications in GBS (Griffin et 1990).

Two sets of experiments are performed. In the first, rats, immunized with peripheral myelin in Freund's adjuvant, are be treated 3-5 days prior to onset of clinical signs and every day thereafter. A control group is untreated. The rats are monitored for the development of clinical signs and the severity scored. Two to three weeks after this, they are perfused and the PNS studied morphologically.

In the second experiment, a tetracycline compound, e.g., minocycline, is given at the onset of clinical signs, and the animals observed thereafter to determine the prevention of progression of the disease and a shortening of the clinical course.

### Example 3

### Other Neurologic Diseases

Inflammation and microglial activation have also been noted to be a significant component of the neuropathology of Parkinson's disease, amyotrophic lateral sclerosis—Lou Gehrig's disease, adrenoleukodystrophy and AIDS encephalopathy. In addition, there has been a recent suggestion that microglia may play a key role in the development of brain abnormalities in patients with schizophrenia (Munn 2000). Based on the data presented above, there seems to be sufficient rationale to consider the compounds of the present invention as a therapy in each of these disorders.

In summary, the present invention provides a method of treating neurologic disorders by administering an effective amount of a tetracycline compound. While not wanting to be bound by any particular theory, it is thought that the anti-inflammatory activity of the compounds of the present invention provides a basis for the compounds' therapeutic value in neurologic disorder.

While the present invention has now been described and exemplified with some specificity, those skilled in the art will appreciate the various modifications, including variations, additions and omissions, that may be made in what has been described. Accordingly, it is intended that these modifications also be encompassed by the present invention and that the scope of the present invention be limited solely be the broadest interpretation that lawfully can be accorded the appended claims.

All patents, publications and references cited herein are hereby fully incorporated by reference. In case of conflict between the present disclosure and incorporated patents, publications and references, the present disclosure should control.

References:

Selkoe, D. J., 1999, Translating cell biology into therapeutic advances in Alzheimer's disease. Nature 399:A23-A30.

Yrjänheikki J., Tikka T., Keinänen, R., Goldsteins, G., Chan, P. H., Koistinaho, J., 1999, A tetracycline derivative, minocycline, reduces inflammation and protects against focal cerebral ischemia with a wide therapeutic window. Proc. Natl. Acad. Sci. USA 96:13496-13500.

Yrjänheikki J., Keinänen, R., Pellikka, M., Hokfelt, T., Koistinaho, J., 1998, Tetracyclines inhibit microglial activation and are neuroprotective in global brain ischemia. Proc. Natl. Acad. Sci. USA 95:15769-15774.

Combs, C. K., Johnson, D. E., Karlo, J. C., Cannady S. B., Landreth, G. E., 2000, Inflammatory mechanisms in Alzheimer's disease: Inhibition of β-amyloid-stimulated proinflammatory responses and neurotoxicity by PPARy agonists. The Journal of Neuroscience 20: 558-567.

Griffin, J. W., Stoll, G., Li, C. Y., Tyor, W., Comblath, D. R., 1990, Macrophage responses in inflammatory demyelinating neuropathies. Ann. Neurol. 27 Suppl. S64-S68.

Matsushima, G. K., Taniike, M., Glimcher, L. H., Grusby, M. J., Frelinger, J. A., Suzuki, K., Ting, J. P-Y, 1994, Absence of MHC class II molecules reduces CNS demyelination, microglial/macrophage infiltration, and twitching in murine globoid cell leukodystrophy. Cell 78:645-656.

Munn, N. A., 2000, Microglia dysfunction in schizophrenia: An integrative theory. Med. Hypotheses 54:198-202.

DeClerck, Y. A., Shimada, H., Taylor, S. M. & Langley, K. E. (1994) Annals N.Y. Acad. Sci. 732, 222-232.

Golub, L. M., Ramamurthy, N. S. & McNamara, T. F. (1991) Crit. Rev. Oral Biol. Med. 2, 297-322.

Golub, L. M., Sorsa, T. & Suomanlainen, K. (1992) Curr. Opin. Dent. 2, 80-90.

Greenwald, R. A. (1994) Annals N.Y Acad. Sci. 732, 181-198.

Greenwald, R. A. & Golub, L.M., eds. (1994) Inhibition of Matrix Metalloproteinases: Therapeutic Potential. Annals N.Y. Acad. Sci Vol. 732, 1-507.

Mitscher, L. A. (1978) The Chemistry of the Tetracycline Antibiotics, Ch. 6, Marcel Dekker, N.Y.

Mohtai, M., Smith, R. L., Schurman, D. J., Taub, Y., Torti, F. M., Hutchinson, N. I., Stetler-Stevenson, W. G. & Goldberg, G. I. (1993) J. Clin. Inv. 92, 179-185.

Ramamurthy, N., Greenwald, R., Moak, S., Scuibba, J., Goren, A., Turner, G., Rifkin, B. & Golub, L. (1994) Annals N.Y. Acad. Sci. 732, 427-430.

Rifkin, B. R., Vernillo, A. T., Golub, L. M. and Ramamurthy, N. S. (1994)

Annals N.Y. Acad. Sci. 732, 165-180.

Tilley, B. C., Alarcon, G. S., Heyse, S. P., Trentham, D. E., Neuner, R., Kaplan, D. A., Clegg, D. O., Leisen, J. C. C., Buckley L., Cooper, S. M., Duncan, H., Pillemer, S. R., Tuttleman, M., Fowler, S. E., Minocycline in rheumatoid arthritis. Ann. Intern. Med. 122:81-89 (1995).

Uitto, V. J., Firth, J. D., Nip, L. & Golub, L. M. (1994) Annals N.Y. Acad. Sci. 732, 140-151.

Yu, L. P., Jr., Smith, G. N., Jr., Brandt, K. D., Myers, S. L., O'Connor, B. L. & Brandt, D. A. (1992) Arthritis Rheum. 35, 1150-1159.

I claim:

1. A method of treating or preventing a disease in a mammal comprising administering an effective amount of a tetracycline compound, the disease being Alzheimers' disease, Guillain Barré syndrome, adreneoleukodystrophy, Parkinson's disease, or amyotrophic lateral sclerosis.

2. A method of downregulating microglia expression in a mammal, comprising administering to the mammal in need thereof an effective amount of a tetracycline compound.

3. A method of inhibiting inflammatory activity associated with microglial activation and production, comprising administering to a mammal in need thereof an effective amount of a tetracycline compound.

4. The method of claim 1 wherein the tetracycline compound is a compound of formula (I) 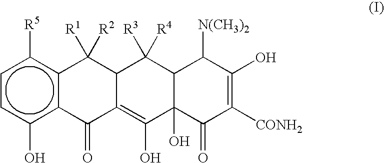
wherein R¹ is CH₃ or OH and R² is H or OH, or R¹ and R² taken together are ═CH₂, R³ and R⁴ are H or OH and R⁵is Cl or N(CH₃)₂.

5. The method of claim 2 wherein the tetracycline compound is a compound of formula (I) 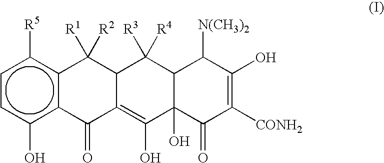
wherein R¹ is CH₃ or OH and R² is H or OH, or R¹ and R² taken together are ═CH₂, R³ and R⁴ are H or OH and R⁵ is Cl or N(CH₃)₂.

6. The method of claim 3 wherein the tetracycline compound is a compound of formula (I) 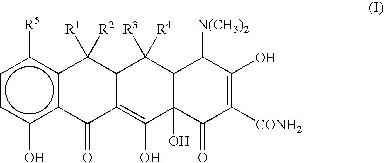
wherein R¹ is CH₃ or OH and R² is H or OH, or R¹ and R² taken together are ═CH₂, R³ and R⁴ are H or OH and R⁵ is Cl or N(CH₃)₂.

7. The method of claim 1 wherein the tetracycline compound is minocycline or doxycycline.

8. The method of claim 3 wherein the effective amount is an antiinflammatory effective amount.

9. The method of claim 1 wherein the tetracycline compound is a non-antibiotic tetracycline compound.

10. The method of claim 2 wherein the tetracycline is minocycline or doxycycline.

11. The method of claim 2 wherein the tetracycline is a non-antibiotic tetracycline.

12. The method of claim 3 wherein the tertracycline compound is minocycline or doxycycline.

13. The method of claim 3 wherein the tetracycline is a non-antibiotic tetracycline.

14. The method of claim 1 wherein the effective amount is about 0.1 mg/kg/day to about 45 mg/kg/day.

15. The method of claim 10 wherein the effective amount is about 1 mg/kg/day to about 18 mg/kg/day.

16. A method of reducing the neurologic symptoms associated with increased expression of microglia or increased microglia cell production in a mammal, the method comprising administering an effective amount of a tetracycline compound to the mammal.

17. The method of claim 3, wherein an antiinflammatory agent is co-administered with the tetracycline compound.

18. The method of claim 12 wherein the tetracycline compound is administered over a period of time until the mammal becomes asymptomatic or the symptoms are reduced by at least 50% compared to pre-treatment symptoms.

19. A pharmaceutical combination comprising a first antiinflammatory agent which is a tetracycline compound and a second antiinflammatory agent.

20. The method of claim 1 wherein the tetracycline compound is administered parenterally, internally or via a controlled release formulation.

21. The method of claim 1 wherein the amount is below the antibiotic effective amount.

22. A method of treating or preventing a neurologic disease in a mammal comprising administering to the mammal suffering therefrom an effective amount of a tetracycline compound of formula (I) 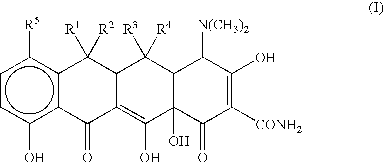
wherein R¹ is CH₃ or OH and R² is H or OH, or R¹ and R² taken together are ═CH₂, R³ and R⁴ are H or OH and R⁵ is Cl or N(CH₃)₂, the disease being Alzheimers' disease, Guillain Barré syndrome, adreneoleukodystrophy, Parkinson's disease, or amyotrophic lateral sclerosis.

23. The method of claim 22 wherein the disease is Alzheimers' disease.

24. The method of claim 22 wherein the disease is Guillain Barré syndrome.

25. The method of claim 22 wherein the disease is adreneoleukodystrophy.

26. The method of claim 22 wherein the disease is Parkinson's disease.

27. The method of claim 22 wherein the disease is amyotrophic lateral sclerosis.

28. The method of claim 22 wherein the amount about 0.1 mg/kg/day to about 45 mg/kg/day.

29. The method of claim 28 wherein the amount is about 0.1 mg/kg/day to about 18 mg/kg/day.

30. The method of claim 22 wherein the amount is sufficient to reduce symptoms of the disease at least 50% compared to pre-treatment symptoms.

31. The method of claim 22 wherein the administering is done for a period of about 2 to 3 weeks or until the mammal becomes asymptomatic of the disease.

32. A method of treating inflammatory conditions associated with Alzheimer's disease, Guillain-Barré syndrome, adrenoleukodystrophy, Parkinson's disease and amytrophic lateral sclerosis in a subject comprising administering to the subject an effect amount of a tetracycline compound in a pharmaceutical carrier.

33. The method of claim 32 wherein the tetracycline compound is a compound of formula (I) 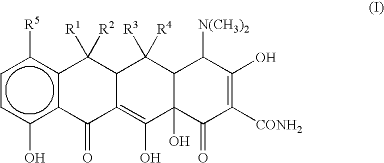
wherein R¹ is CH₃ or OH and R² is H or OH, or R¹ and R² taken together are ═CH₂, R and R⁴ are H or OH and R⁵ is Cl or N(CH₃).

34. The method of claim 32 wherein the tetracycline compound is minocycline or doxycycline.

35. The method of claim 32 wherein the tetracycline compound is a non-antibiotic tetracycline compound.

36. The method of claim 32 wherein the effective amount is about 0.1 to about 45 mg/kg/day.

37. The method of claim 36 wherein the effective amount is about 1 to about 18 mg/kg/day.

38. The method of claim 32 wherein the effective amount is sufficient to reduce symptoms of the inflammatory condition at least 50% compared to pre-treatment symptoms.

39. A method of treating inflammatory conditions associated with Alzheimer's disease, adrenoleukodystrophy, Parkinson's disease and amytrophic lateral sclerosis in a subject comprising administering to the subject an effect amount of a tetracycline compound in a pharmaceutical carrier.

40. A method of modulating the effects of inflammatory conditions associated with Alzheimer's disease, Guillain-Barré syndrome, adrenoleukodystrophy, Parkinson's disease and amytrophic lateral sclerosis in a subject comprising administering to the subject an effect amount of a tetracycline compound in a pharmaceutical carrier.

41. A combined pharmaceutical preparation, comprising a tetracycline compound of formula (I) 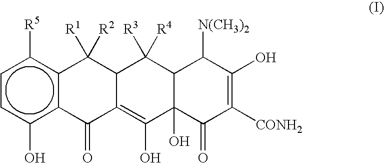
and an anti-inflammatory agent, the preparation being adapted for administration on a daily basis to a subject having Alzheimer's disease, Guillain Barré syndrome, Parkinson's disease, adrenoleukodystrophy or amyotrophic lateral sclerosis..

42. A pharmaceutical packaging comprising (i) a plurality of containers therein, at least one of the containers containing a tetracycline compound of formula (I) 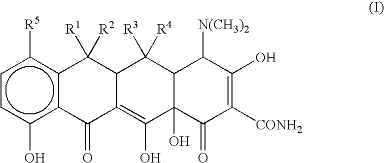
and at least one of the containers containing an agent which is an anti-inflammatory agent or a free radical scavenger, and (ii) instructions for co-administering the tetracycline compound and anti-inflammatory agent to a subject having Alzheimer's disease, Guillain Barré syndrome, Parkinson's disease, adrenoleukodystrophy or amyotrophic lateral sclerosis.

43. A pharmaceutical packaging in accordance with claim 42, wherein the tetracycline is provided in a controlled release formulation.

44. A pharmaceutical packaging in accordance with claim 42, wherein the agent is an anti-inflammatory agent.

45. A pharmaceutical packaging in accordance with claim 42, wherein the agent is a free radical scavenger.

46. A pharmaceutical packaging in accordance with claim 42, further comprising both an anti-inflammatory agent and a free radical scavenger.
